(19) United States
(12) Patent Application Publication
(10) Pub. No.: US 2025/0367335 A1
(43) Pub. Date: Dec. 4, 2025

(54) Wearable Electronic Device Sterilization

(71) Applicant: Sensor Electronic Technology, Inc., Columbia, SC (US)

(72) Inventor: Piyush Sharma, Columbia, SC (US)

(21) Appl. No.: 19/220,708

(22) Filed: May 28, 2025

Related U.S. Application Data
(60) Provisional application No. 63/654,425, filed on May 31, 2024.

(51) Int. Cl. A61L 2/10; A61L 2/24; F16M 11/22; G06F 1/18

(52) U.S. Cl. CPC A61L 2/10; A61L 2/24; F16M 11/22; G06F 1/18; A61L 2202/11; A61L 2202/14; A61L 2202/16

(57) ABSTRACT

A stand for a wearable electronic device, such as a virtual reality headset, can include a platform configured to support the wearable electronic device which can include a treatment surface having an ultraviolet transparent structure with an outer surface configured to conform to a shape of a contact surface of the wearable electronic device. The stand can include one or more ultraviolet light sources configured to direct ultraviolet radiation through the outer surface of the transparent structure and onto the contact surface of the wearable electronic device. The stand can further include a charging interface configured to enable a battery of the wearable electronic device to be charged while the wearable electronic device is located on the platform.

### REFERENCE TO RELATED APPLICATIONS

The current application claims the benefit of U.S. Provisional Application No. 63/654,425, filed on 31 May 2024, which is hereby incorporated by reference.

### TECHNICAL FIELD

The disclosure relates generally to wearable electronic devices such as headsets, and more particularly, to sterilization of such electronic devices, such as virtual reality headsets.

### BACKGROUND ART

In the rapidly evolving landscape of virtual reality (VR) technology, the demand for VR headsets in the United States has surged, reaching a valuation of USD 2.53 billion in 2022. Projections indicate substantial growth, with expectations to reach USD 36.32 billion by 2032, showcasing a robust Compound Annual Growth Rate (CAGR) of 30.60% from 2023 to 2032. Amidst this flourishing market, the predominant approach to VR headset maintenance involves standalone charging stands. To date, these stands lack an integrated solution to address hygiene concerns associated with VR headset use.

The market context underscores the significance of innovations that cater to the burgeoning VR headset industry. Current VR headset maintenance practices fall short in comprehensive hygiene solutions, exposing users to potential health risks from the accumulation of face oils, germs, and bacteria on headset surfaces. In particular, current charging stands often do not provide any hygienic functionality. Standalone ultraviolet (UV) sterilization devices exist, but they require separate use by individuals.

### SUMMARY OF THE INVENTION

The inventor recognizes a need to address hygiene concerns associated with VR headset use. Standalone UV sterilization devices lack specificity for the unique design of VR headsets, potentially leading to incomplete sterilization. Traditional charging stands miss the opportunity for holistic integration of both charging and sterilization functionalities, resulting in a fragmented maintenance approach.

Aspects of the invention provide a stand for a wearable electronic device, such as a virtual reality headset, which can include a platform configured to support the wearable electronic device. The platform can include a treatment surface having an ultraviolet transparent structure with an outer surface configured to conform to a shape of a contact surface of the wearable electronic device. The stand can include one or more ultraviolet light sources configured to direct ultraviolet radiation through the outer surface of the transparent structure and onto the contact surface of the wearable electronic device. The stand can further include a charging interface configured to enable a battery of the wearable electronic device to be charged while the wearable electronic device is located on the platform.

Embodiments of the invention described herein are directed to a multifunctional stand intricately equipped with a specialized UV LED system. The system can be configured to achieve targeted sterilization of the fabric or rubberized materials that are directly in contact with the user's skin during use of a wearable electronic device configured to be worn on a head of a user, such as a VR headset. Importantly, by fostering a hygienic environment, embodiments of the invention can enable users to save significantly on fabric replacement costs for the headset, extending the lifespan of the VR headset components. Furthermore, the same VR headset can be used by multiple individuals in a more hygienic fashion.

Embodiments can seamlessly integrate both charging and UV sterilization functionalities within a purpose-built stand, embodiments of the invention can address limitations of existing solutions and enhance user convenience, while providing a cost-saving measure. The unique combination of features presented in embodiments can provide a user-centric contribution to the dynamic landscape of wearable electronic device technology, and in particular, virtual reality headset technology, aligning with the escalating demands of the growing VR headset market in the United States.

A first aspect of the invention provides a stand for a wearable electronic device, the stand comprising: a platform configured to support the wearable electronic device, wherein the platform includes a treatment surface having an ultraviolet transparent structure with an outer surface configured to conform to a shape of a contact surface of the wearable electronic device; and at least one ultraviolet light source configured to direct ultraviolet radiation through the outer surface of the transparent structure and onto the contact surface of the wearable electronic device.

A second aspect of the invention provides a stand for a wearable electronic device, the stand comprising: a base configured for placement on a surface; a support member secured to the base; a platform configured to support the wearable electronic device, wherein the support member is secured to and supports the platform above the surface, wherein the platform includes a treatment surface having an ultraviolet transparent structure with an outer surface configured to conform to a shape of a contact surface of the wearable electronic device; at least one ultraviolet light source configured to direct ultraviolet radiation through the outer surface of the transparent structure and onto the contact surface of the wearable electronic device; and at least one charging interface configured to enable a battery of the wearable electronic device to be charged while the wearable electronic device is located on the platform.

A third aspect of the invention provides a stand for a wearable electronic device, the stand comprising: a platform configured to support the wearable electronic device, wherein the platform includes a treatment surface having an ultraviolet transparent structure with an outer surface configured to conform to a shape of a contact surface of the wearable electronic device; at least one ultraviolet light source configured to direct ultraviolet radiation through the outer surface of the transparent structure and onto the contact surface of the wearable electronic device; a controller configured to operate the at least one ultraviolet light source to treat the wearable electronic device; and at least one sensing device configured to acquire location data corresponding to a presence or an absence of the contact surface of the wearable electronic device on the outer surface of the transparent structure, wherein the controller is configured to operate the at least one ultraviolet light source so that the at least one ultraviolet light source is off when the location data corresponds to an absence of the contact surface.

The illustrative aspects of the invention are designed to solve one or more of the problems herein described and/or one or more other problems not discussed.

### BRIEF DESCRIPTION OF THE DRAWINGS

These and other features of the disclosure will be more readily understood from the following detailed description of the various aspects of the invention taken in conjunction with the accompanying drawings that depict various aspects of the invention.

FIGS. 1A-1C show illustrative virtual reality headsets, which can be concurrently charged and treated using a device described herein.

FIGS. 2A-2C show various views of an illustrative charging and treatment stand according to an embodiment.

FIG. 3 shows an illustrative virtual reality headset mounted on an illustrative charging and treatment stand according to an embodiment.

FIG. 4 shows a front schematic view of an illustrative charging and treatment stand according to an embodiment.

FIG. 5 shows an illustrative schematic representation of a system for treating a virtual reality headset according to embodiments.

It is noted that the drawings may not be to scale. The drawings are intended to depict only typical aspects of the invention, and therefore should not be considered as limiting the scope of the invention. In the drawings, like numbering represents like elements between the drawings.

### DETAILED DESCRIPTION OF THE INVENTION

As indicated above, aspects of the invention provide a stand for a wearable electronic device, such as a virtual reality headset, which can include a platform configured to support the wearable electronic device. The platform can include a treatment surface having an ultraviolet transparent structure with an outer surface configured to conform to a shape of a contact surface of the wearable electronic device. The stand can include one or more ultraviolet light sources configured to direct ultraviolet radiation through the outer surface of the transparent structure and onto the contact surface of the wearable electronic device. The stand can further include a charging interface configured to enable a battery of the wearable electronic device to be charged while the wearable electronic device is located on the platform.

Embodiments can be directed to a system configured to treat (e.g., sterilize) fabric or rubberized materials of a wearable electronic device, such as a headset, e.g., a virtual reality (VR) headset, which can come directly in contact with the user's skin during use of the device. As VR applications become more ubiquitous, opportunities will exist for VR environments in which the headset may be used by multiple people. For example, public places, such as exercise facilities, entertainment venues, amusement parks, museums, etc., may incorporate an ability to use VR in order to enhance the experience of their visitors. For some such applications, Embodiments of the invention can enable owners and operators of such VR environments to save significantly on fabric replacement costs for the headset, extending the lifespan of the VR headset components.

By seamlessly integrating both charging and UV sterilization functionalities within a purpose-built stand, embodiments of the invention can address limitations of existing solutions and enhance user convenience, while providing a cost-saving measure.

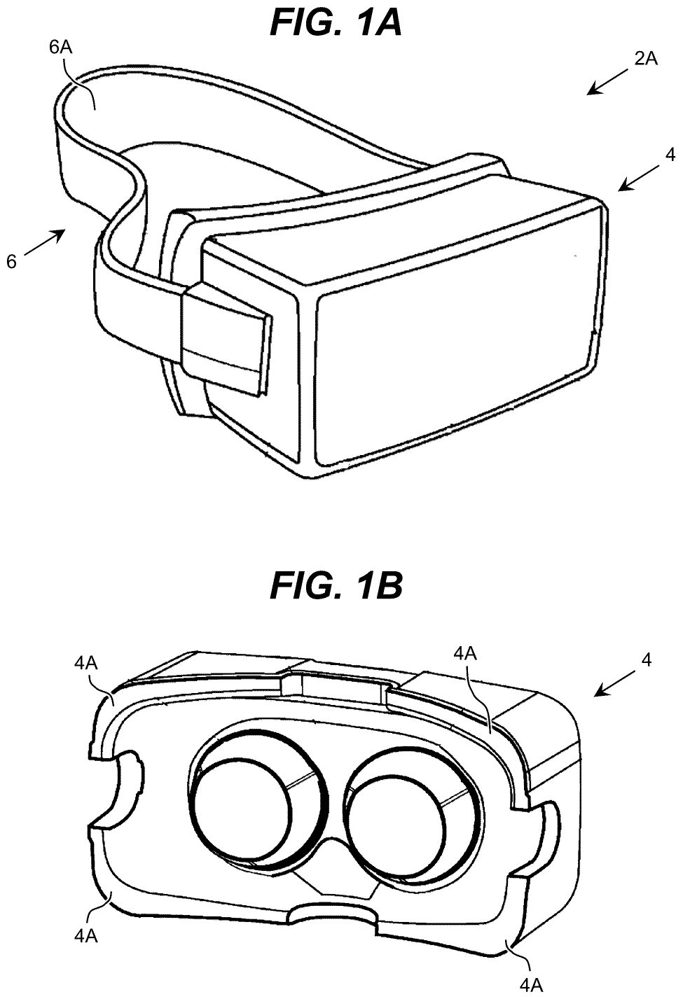

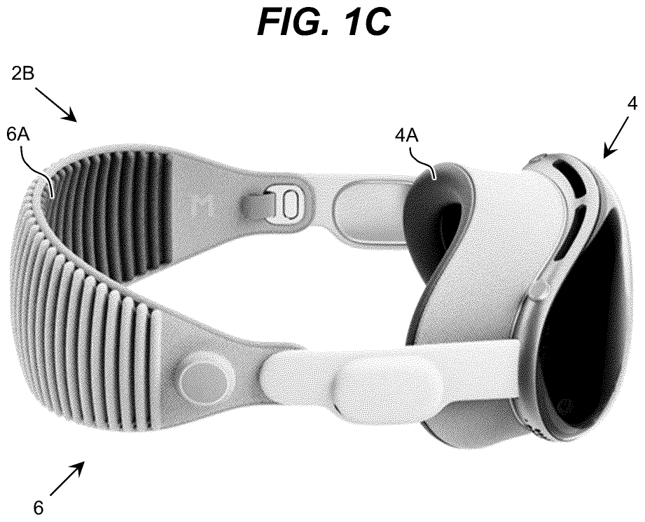

Turning to the drawings, FIGS. 1A-1C show illustrative virtual reality headsets 2A, 2B, which can be concurrently charged and treated using a device described herein. As illustrated, a virtual reality headset 2A, 2B can typically include a front body 4, which includes electronics and structures for generating and presenting a virtual reality display to a user. The front body 4 is secured to a user's head via one or more bands 6.

During use, the front body 4 is held against a user's head via contact surfaces 4A. Each contact surface 4A can comprise a fabric, rubber, etc., exterior surface and can include padding for user comfort. In general, the contact surfaces 4A are the only portion of the front body 4 that contact the user's head and skin. Similarly, an inner surface 6A of the band 6, particularly the inner surface located in the back of the head, will contact the user's head in order to hold the front body 4 in place during use.

Embodiments provide a stand that can treat some or all of the surfaces of the headset 2A, 2B that come in direct contact with the user's head during use. For example, embodiments of the stand can treat some or all of the contact surfaces 4A on the front body 4, some or all of the inner surface 6A of the band 6, and/or the like.

Further embodiments of the stand also enable the headset to be recharged. To this extent, embodiments of the stand can include interfaces that enable a battery in a headset 2A, 2B to be recharged.

Embodiments of the stand can include a charging system and a treatment system, which can concurrently charge and treat a headset 2A, 2B mounted thereto.

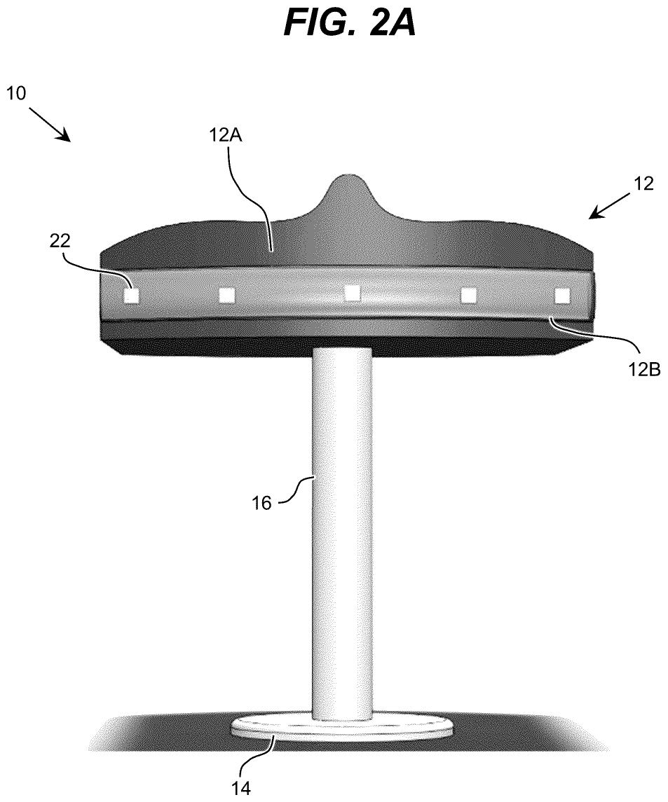

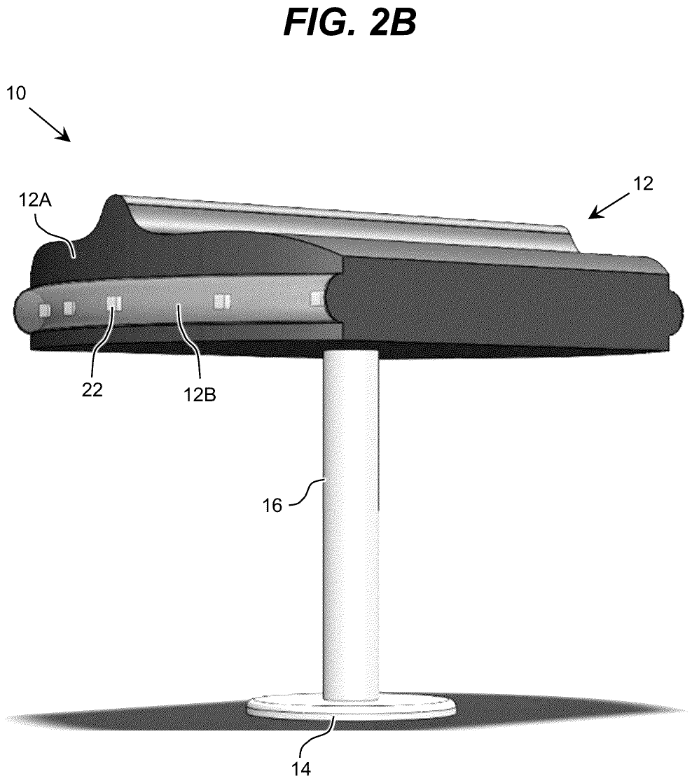

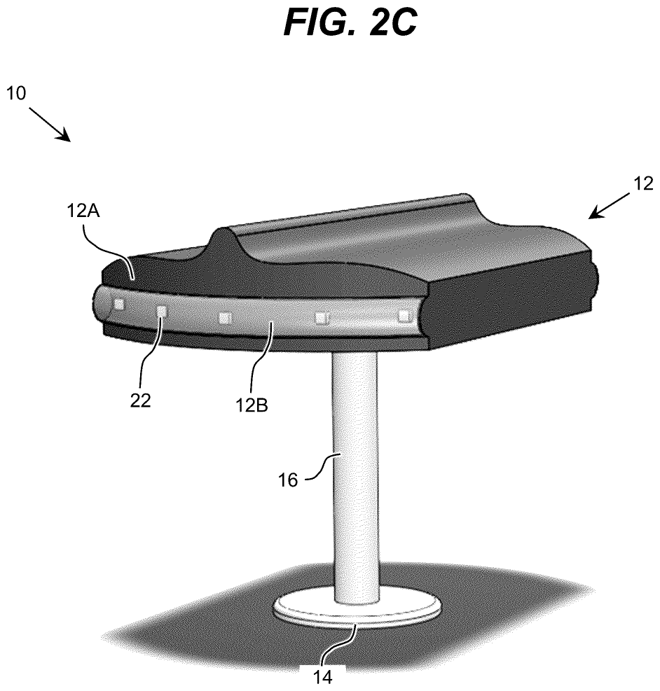

FIGS. 2A-2C show various views of an illustrative charging and treatment stand 10 according to an embodiment. As illustrated, the stand 10 includes a headset platform 12, a base 14, and a support member 16, which is connected to the base 14, and supports the headset platform 12. It is understood that the illustrated configuration is only illustrative of various possible embodiments of charging and treatment stands 10 described herein.

For example, embodiments of a charging and treatment stand 10 can include a different shaped base 14, a different base configuration (e.g., multiple legs), two or more support members 16, support member(s) having a different shape, one or more adjustable support members 16 (e.g., height adjustable, adjustable angle, etc.), a different shaped headset platform 12, etc. As illustrated, the charging and treatment stand 10 can be configured for placement on a surface. In embodiments, the charging and treatment stand 10 can be configured to be mounted to a structure (e.g., mounted to a horizontal structure, such as a desktop, mounted to vertical structure, such as a wall, etc.) using any mounting solution.

In embodiments, each of the headset platform 12, base 14, and support member 16 can be constructed from a material configured to provide durability and/or lightweight properties. In more particular embodiments, the headset platform 12, base 14, and/or support member 16 can be constructed from a plastic, such as acrylonitrile butadiene styrene (ABS), polycarbonate, a reinforced plastic, and/or the like. In embodiments, the headset platform 12, base 14, and/or support member 16 can be constructed from a metal, such as aluminum. To increase strength, embodiments of the support member 16 can comprise steel. Embodiments of the base 14 can include one or more pads on the bottom surface, which can be formed of rubber, silicone, and/or the like.

The headset platform 12, base 14, and support member 16 can be secured to each other using any permanent or selectively removable securing mechanism. For example, the support member 16 can be secured to the headset platform 12 and/or the base 14 using one or more of a threaded connection, an interference fit, glue, solder, a securing pin or ratchet, etc.

Regardless, the headset platform 12 can include various components to enable one or more surfaces of a headset 2A, 2B to be treated. For example, the headset platform 12 can include a treatment surface 12A which is configured to treat one or more surfaces of a headset 2A, 2B secured thereon. In embodiments, the treatment surface 12A comprises one or more ultraviolet transparent structures 12B that can be configured to transmit and/or direct ultraviolet light onto the surface(s) of the headset 2A, 2B to be treated.

In embodiments, an ultraviolet transparent structure 12B has a curved outer surface, which is configured to generally conform to a typical shape for contact surfaces 4A of a headset 2A, 2B. To this extent, as shown most clearly in FIG. 2C, the ultraviolet transparent structure 12B can be curved both laterally and vertically. The transparent structure 12B can be formed from any suitable material that is transparent to the ultraviolet light. Illustrative materials for the ultraviolet transparent structure 12B include ultraviolet resistant plastics (e.g., polycarbonate, acrylic, etc.). The ultraviolet transparent structure 12B can be secured to the headset platform 12 using any solution, including one or more screws, bolts, or other fasteners, glue, etc. To provide protection against dust or moisture penetrating a seal, the junction can include a sealant, such as silicone, rubber, and/or the like. In embodiments, the outer curve of the ultraviolet transparent structure 12B has lateral and vertical sizes and curves such that all of the contact surfaces 4A of a headset 2A, 2B contact the outer surface of the ultraviolet transparent structure 12B when the headset 2A, 2B is secured to the headset platform 12 using the band 6.

In embodiments, the ultraviolet light can be generated by a set of ultraviolet light sources 22. In some embodiments, the ultraviolet light sources 22 can be mounted on the headset platform 12 and can be configured to emit ultraviolet light that is directed through the ultraviolet transparent structure 12B and onto a surface of the headset 2A, 2B to be treated. In more particular embodiments, the headset platform 12 includes multiple ultraviolet light sources 22, e.g., arranged laterally and/or horizontally along the treatment surface 12A.

In embodiments, the ultraviolet light has one or more peak wavelengths. In more particular embodiments, the ultraviolet light includes one or more peak wavelengths in the ultraviolet-C range of ultraviolet radiation. In still more particular embodiments, the ultraviolet light includes one or more peak wavelengths in a wavelength range between approximately 200 and approximately 360 nanometers. In more particular embodiments, the wavelength range is between approximately 200 and approximately 280 nanometers. In still more particular embodiments, the wavelength range is between approximately 260 and 270 nanometers.

In embodiments, one or more of the ultraviolet light sources 22 comprises an ultraviolet light emitting diode (LED). In embodiments, multiple ultraviolet light sources 22 can emit ultraviolet light having similar or comparable peak wavelengths. In other embodiments, multiple ultraviolet light sources 22 can emit ultraviolet light having different peak wavelengths.

In embodiments, the ultraviolet light sources 22 can include light directing structures that are configured to guide ultraviolet light emitted from an ultraviolet light source 22 located at a distinct location, such as within an interior region of the headset platform 12. For example, an ultraviolet light source 22 can include a wave guide structure, such as an optical fiber, one or more mirrors, one or more lenses, and/or the like, each of which is configured to direct ultraviolet light for emission from the ultraviolet light source 22 and through the ultraviolet transparent structure 12B. In embodiments, the light directing structures can include fixed structures and/or structures configured to be operated to enable focused and comprehensive treatment of the surface(s) of the headset 2A, 2B in any of various locations.

In embodiments, one or more ultraviolet light sources 22 can be included on two or more surfaces of the headset platform 12. For example, in embodiments, one or more ultraviolet light sources 22 can be included on opposing sides of the headset platform 12. In this configuration, the headset platform 12 can enable the headset 2A, 2B to be mounted and treated while facing either direction. In embodiments, such a headset platform 12 can enable treatment of both the contact surfaces 4A as well as the inner surface 6A of the band 6 located opposite the contact surfaces 4A.

In embodiments, the opposing sides of the headset platform 12 can comprise ultraviolet transparent structures 12B having different shapes and/or ultraviolet light sources 22 having different configurations, each of which is designed to facilitate treatment of the contact surfaces 4A and inner surface 6A, respectively. For example, as discussed herein, the shape of the respective ultraviolet transparent structures 12B can be configured to ensure that the surface(s) being treated are in direct contact with the outer surface of the corresponding ultraviolet transparent structure 12B. In a more particular embodiment, an ultraviolet transparent structure 12B configured to treat the inner surface 6A can be laterally curved, while being substantially straight in a vertical direction to facilitate good contact for all of the inner surface 6A of the band 6 in the treatment area of the ultraviolet transparent structure 12B.

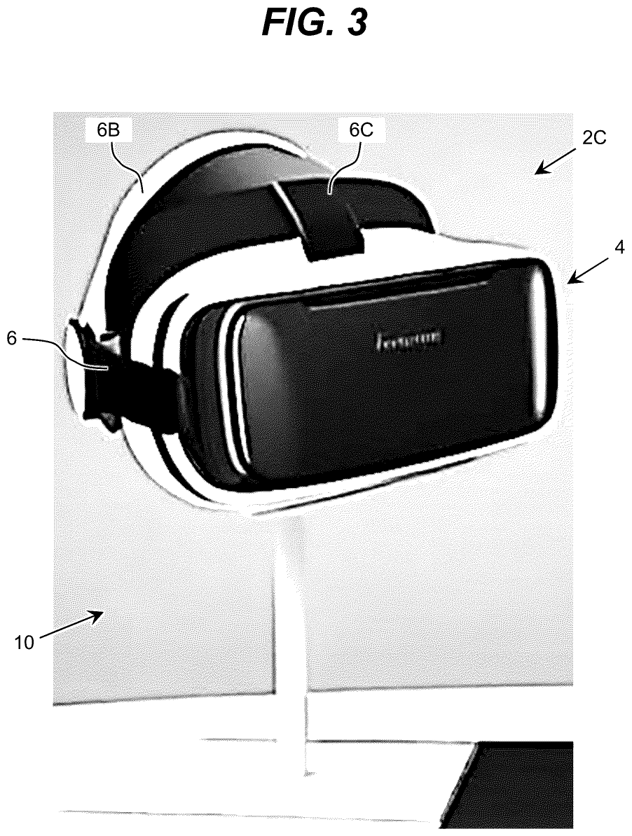

It is understood that a headset platform 12 can include any number of treatment surfaces, each of which can be configured to treat one or more surfaces that come in contact with a user during use of a corresponding headset. To this extent, FIG. 3 shows an illustrative virtual reality headset 2C mounted on an illustrative charging and treatment stand 10 according to an embodiment. In this case, in addition to a strap 6, which is configured to be placed around the head of the user, the headset 2C includes a strap 6B that is configured to go over a top of the user's head from one side to the other, and a strap 6C that is configured to go over the top of the user's head from front to back. In embodiments, a headset platform 12 described herein can include treatment surfaces that are configured to treat some or all of the inner surfaces of one or more of such straps 6, 6B, 6C. To this extent, the headset platform 12 can include surfaces that are oriented and shaped such that the straps 6, 6B, 6C are secured tightly thereto when the headset 2C is properly placed on the headset platform 12. Such surfaces can be transparent to ultraviolet radiation, include one or more ultraviolet radiation sources, and/or one or more additional features as described herein.

It is understood that a charging and treatment stand 10 described herein can include various components to facilitate treatment and charging of a headset mounted thereon, as well as safe operation of the ultraviolet light sources 22.

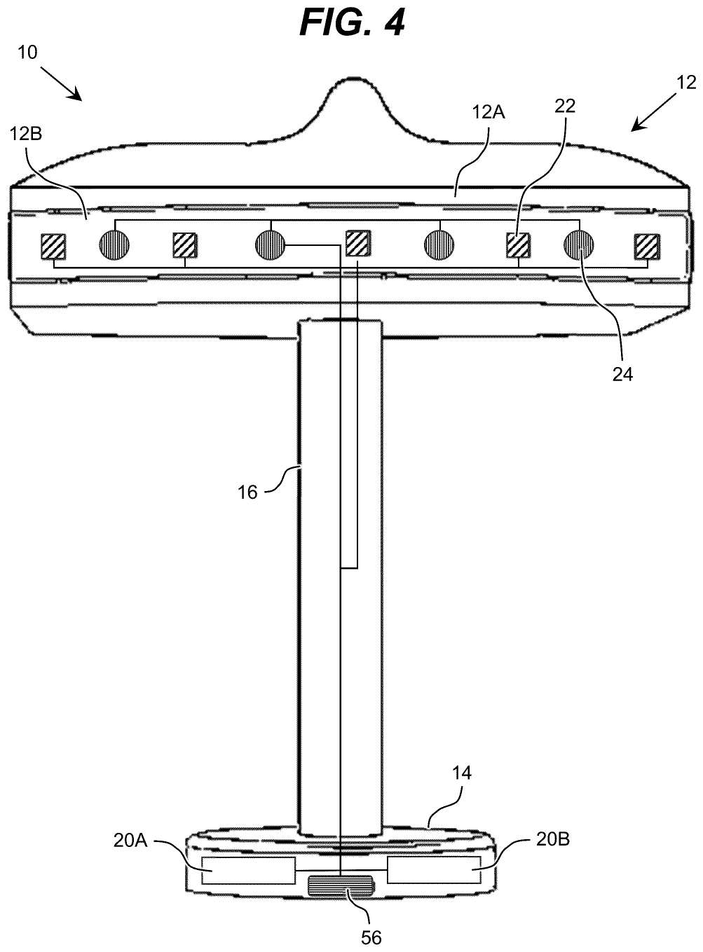

FIG. 4 shows a front schematic view of an illustrative charging and treatment stand 10 according to an embodiment. As illustrated, the charging and treatment stand 10 can include one or more computing devices 20A, 20B for operating the various components to treat and/or charge a headset. While the charging and treatment stand 10 is shown including two computing devices 20A, 20B located in the base 14, it is understood that a charging and treatment stand 10 can include any number of one or more computing devices, each of which can be located anywhere on the charging and treatment stand 10. In embodiments, all devices can be located in the headset platform 12, which can enable the headset platform 12 to be operated without the base 14 and support member 16. Additionally, embodiments of the charging and treatment stand 10 can include a computing device 20A, 20B configured to communicate with an external computing device (e.g., a personal computer, a laptop, a mobile device, etc.), which can be configured to monitor and/or manage one or more aspects of the operation of the charging and treatment stand 10.

Regardless, the charging and treatment stand 10 can include ultraviolet light sources 22 and one or more sensing devices 24. Each sensing device 24 can be configured to acquire data used in conjunction with operation of the charging and treatment stand 10 using any solution. A sensing device 24 described herein may include an emitter and a sensor, which are operated in conjunction with one another in order to acquire the desired data.

Embodiments of the sensing devices 24 can include one or more sensors, such as proximity sensors, pressure sensors, and/or the like, which are configured to acquire data corresponding to a presence or an absence. e.g., a placement of and/or a location of one or more surfaces of a headset mounted to the charging and treatment stand 10. Illustrative proximity sensors comprise infrared sensors, capacitive sensors, ultrasonic sensors, laser sensors, optical sensors, etc. In embodiments, a pressure sensor can provide data regarding the location and/or weight of the headset, which can be processed to ensure proper placement of the headset.

In this case, a computing device 20A, 20B can receive data from a sensing device 24 to determine a location of a surface to be treated and adjust operation of one or more ultraviolet light sources 22 accordingly. Such operation can include, for example, turning an ultraviolet light source 22 on or off, directing the ultraviolet light emitted from an ultraviolet light source 22 to a location of the surface to be treated, etc. Embodiments enable the charging and treatment stand 10 to only direct ultraviolet light in locations to be treated, avoiding the possibility of damage or harm resulting from inadvertent exposure to ultraviolet radiation.

Embodiments of the sensing devices 24 can include one or more sensors configured to acquire other data for use in treating a headset. For example, a sensing device 24 can be configured to acquire data regarding a presence or amount of a contaminant on a surface of a headset mounted to the charging and treatment stand 10. Such a sensing device 24 can detect fluorescence emitted by a contaminant, a surface appearance caused by a contaminant, and/or the like.

Embodiments of the charging and treatment stand 10 can adjust one or more aspects of treatment of the surface(s) of a headset 2A, 2B. For example, different wavelengths, doses, intensities, durations, and/or the like, of ultraviolet radiation used in a treatment can be selected based on the type of surface being treated, whether any known or likely contamination is present, in response to a selection by a user, in response to a time since a previous treatment, etc. The amount of time, power, dose, etc., required for treatment will vary based on the materials used in the charging and treatment stand 10, a number of ultraviolet light sources 22, a maximum possible radiant flux generated by the ultraviolet light sources 22, the material being treated, a target contaminant, etc.

Regardless, in embodiments, an exposure time can be between approximately 5 to approximately 10 minutes; an ultraviolet intensity can be in a range between approximately 1 and approximately 5 mW/cm²; a distance between an ultraviolet light source 22 and a surface to be treated can be in a range between approximately 1 to approximately 5 cm; etc.

As illustrated, the charging and treatment stand 10 can include wiring, which enables a computing device 20A, 20B to operate the various ultraviolet light sources 22 and sensing devices 24. It is understood that such wiring can enable operation of each device 22, 24 individually and/or collective operation of two or more devices 22, 24.

As discussed herein, the charging and treatment stand 10 also can enable a battery of a headset mounted thereto to be charged. To this extent, the charging and treatment stand 10 also is shown including a charging interface 56. The charging interface 56 can comprise any type of interface for enabling the battery of a headset to be charged. For example, the charging interface 56 can comprise a universal serial bus (USB) port. However, it is understood that the location and type of charging interface 56 can be varied and includes any type of wired or wireless charging interface. A computing device 20A, 20B can manage the charging using any solution.

In embodiments in which the charging interface 56 also enables the exchange of data, a computing device 20A, 20B can communicate with a computing device on the headset to provide and/or acquire various data. For example, a computing device 20A, 20B can receive information regarding a unique identifier for the headset, a time since a previous charge of the battery, an amount of time the headset was in use for one or more previous users, a time since a previous treatment of the headset, etc. Furthermore, the computing device 20A, 20B can provide data for use by and/or storage on the headset, such as data corresponding to a unique identifier for the charging and treatment stand 10, a type of treatment performed, a timestamp for the treatment, etc. In this manner, a user, a headset, a charging and treatment stand 10, and/or the like, can use the data to schedule a future treatment, adjust one or more aspects of a treatment, determine no treatment is required, etc.

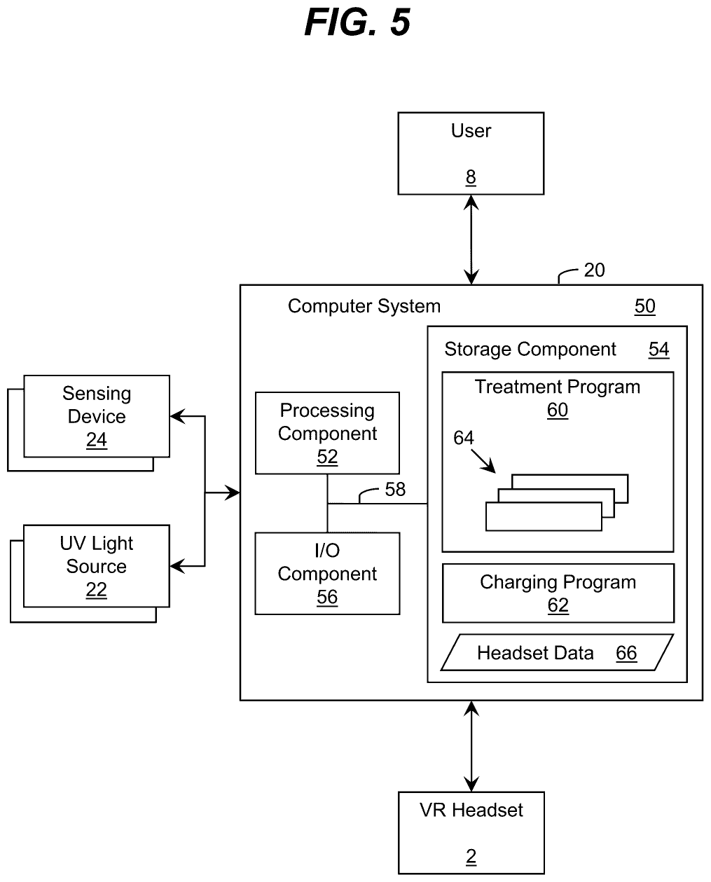

FIG. 5 shows an illustrative schematic representation of a system for treating a virtual reality headset 2 according to embodiments. The system includes a computing device 20, which is shown implemented as a computer system 50 that can perform a process described herein in order to charge and/or treat a headset 2. In particular, the computer system 50 is shown including a treatment program 60 and a charging program 62, which make the computer system 50 operable to charge and/or treat the headset 2 by performing a process described herein.

The computer system 50 is schematically illustrated as including a processing component 52 (e.g., one or more processors), a storage component 54 (e.g., a storage hierarchy), an input/output (I/O) component 56 (e.g., one or more I/O interfaces and/or devices), and a communications pathway 58. In general, the processing component 52 executes program code, such as the treatment program 60 and the charging program 62, which is at least partially fixed in storage component 54. While executing program code, the processing component 52 can process data, which can result in reading and/or writing transformed data from/to the storage component 54 and/or the I/O component 56 for further processing. The pathway 58 provides a communications link between each of the components in the computer system 50.

The I/O component 56 can comprise one or more I/O devices, which enable data exchange between the computer system 50 and a VR headset 2, a user 8, etc. To this extent, the I/O component 56 can include human I/O devices that enable a human user to interact with the computer system 50 and/or one or more communications devices to enable another computing device (e.g., a portable computing device of a human user, such as a mobile phone executing an app) to communicate with the computer system 50 using any type of communications link. To this extent, the treatment program 60 and/or charging program 62 can manage a set of interfaces (e.g., graphical user interface(s), application program interface, and/or the like) that enable human and/or other computer systems to interact with the treatment program 60, the charging program 62, and/or the headset data 66. Furthermore, the treatment program 60 and the charging program 62 can manage (e.g., store, retrieve, create, manipulate, organize, present, etc.) the headset data 66 using any solution.

The I/O component 56 also can include one or more interfaces that enable the computer system 20 to receive data from and/or control the operation of various devices, such as the ultraviolet light sources 22, the sensing devices 24, etc. To this extent, the treatment program 60 and/or the charging program 62 can be configured to store data received from one or more of the devices 22, 24 as headset data 66 and/or use headset data 66 to adjust the operation of one or more of the devices 22, 24 as described herein.

In any event, the computer system 50 can comprise one or more general purpose computing articles of manufacture (e.g., computing devices) capable of executing program code, such as the treatment program 60 and/or the charging program 62, installed thereon. As used herein, it is understood that “program code” means any collection of instructions, in any language, code or notation, that cause a computing device having an information processing capability to perform a particular action either directly or after any combination of the following: (a) conversion to another language, code or notation; (b) reproduction in a different material form; and/or (c) decompression. To this extent, the treatment program 60 and/or the charging program 62 can be embodied as any combination of system software and/or application software.

Furthermore, the treatment program 60 and/or the charging program 62 can be implemented using a set of modules 64. In this case, a module 64 can enable the computer system 50 to perform a set of tasks used by the treatment program 60 and/or the charging program 62, and can be separately developed and/or implemented apart from other portions of the treatment program 60 and/or the charging program 62. As used herein, the term “component” means any configuration of hardware, with or without software, which implements the functionality described in conjunction therewith using any solution, while the term “module” means program code that enables a computer system 50 to implement the actions described in conjunction therewith using any solution. Regardless, it is understood that two or more components, modules, and/or systems may share some/all of their respective hardware and/or software. Furthermore, it is understood that some of the functionality discussed herein may not be implemented or additional functionality may be included as part of the computer system 50.

When the computer system 50 comprises multiple computing devices, each computing device can have only a portion of the treatment program 60 and/or the charging program 62 fixed thereon (e.g., one or more modules 62). In embodiments, the computer system 50 can comprise a computing unit located within the charging and treatment stand 10 and a portable computing unit, such as a mobile phone, which is executing an app installed thereon for enabling a user 8 to monitor, evaluate, manage, and/or the like, operation of the charging and treatment stand 10.

However, it is understood that the computer system 50 and the treatment program 60 and/or the charging program 62 are only representative of various possible equivalent computer systems that may perform a process described herein. To this extent, in other embodiments, the functionality provided by the computer system 50 and the treatment program 60 and/or the charging program 62 can be at least partially implemented by one or more computing devices that include any combination of general and/or specific purpose hardware with or without program code. In each embodiment, the hardware and program code, if included, can be created using standard engineering and programming techniques, respectively.

Regardless, when the computer system 50 includes multiple computing devices, the computing devices can communicate over any type of communications link. Furthermore, while performing a process described herein, the computer system 50 can communicate with one or more other computer systems and/or devices using any type of communications link. In either case, the communications link can comprise any combination of various types of optical fiber, wired, and/or wireless links; comprise any combination of one or more types of networks; and/or utilize any combination of various types of transmission techniques and protocols.

As used herein, unless otherwise noted, the term “set” means one or more (i.e., at least one) and the phrase “any solution” means any now known or later developed solution. The singular forms “a,” “an,” and “the” include the plural forms as well, unless the context clearly indicates otherwise. Additionally, the terms “comprises,” “includes,” “has,” and related forms of each, when used in this specification, specify the presence of stated features, but do not preclude the presence or addition of one or more other features and/or groups thereof.

As also used herein, a layer is a transparent layer when the layer allows at least ten percent of radiation having a target wavelength, which is radiated at a normal incidence to an interface of the layer, to pass there through. Furthermore, as used herein, a layer is a reflective layer when the layer reflects at least ten percent of radiation having a target wavelength, which is radiated at a normal incidence to an interface of the layer. In an embodiment, the target wavelength of the radiation corresponds to a wavelength of radiation emitted or sensed (e.g., peak wavelength+/−five nanometers) by an active region of an optoelectronic device during operation of the device. For a given layer, the wavelength can be measured in a material of consideration and can depend on a refractive index of the material.

It is understood that, unless otherwise specified, each value is approximate and each range of values included herein is inclusive of the end values defining the range. Terms of degree such as “generally,” “substantially,” “about,” and “approximately” as used herein mean a reasonable amount of deviation of the modified term such that the end result is not significantly changed. For example, these terms can be construed as including a deviation of at least +/−0.5% of the modified term if this deviation would not negate the meaning of the word it modifies. In a more particular example, the term “approximately” is inclusive of values within +/−ten percent of the stated value, while the term “substantially” is inclusive of values within +/−five percent of the stated value when these deviations would not negate the meaning of the word each term modifies. Unless otherwise stated, two values are “similar” when the amount of deviation between the two values does not significantly change the result. In a more particular example, two values are similar when the smaller value is within +/−twenty-five percent of the larger value. A value, y, is on the order of a stated value, x, when the value y satisfies the formula 0.1x≤y≤10x.

The foregoing description of various aspects of the invention has been presented for purposes of illustration and description. It is not intended to be exhaustive or to limit the invention to the precise form disclosed, and obviously, many modifications and variations are possible. Such modifications and variations that may be apparent to an individual in the art are included within the scope of the invention as defined by the accompanying claims.

What is claimed is:

1. A stand for a wearable electronic device, the stand comprising:
a platform configured to support the wearable electronic device, wherein the platform includes a treatment surface having an ultraviolet transparent structure with an outer surface configured to conform to a shape of a contact surface of the wearable electronic device; and
at least one ultraviolet light source configured to direct ultraviolet radiation through the outer surface of the transparent structure and onto the contact surface of the wearable electronic device.

2. The stand of claim 1, wherein the outer surface of the transparent structure is curved both laterally and vertically.

3. The stand of claim 1, wherein the transparent structure is secured to and is formed of a different material than the treatment surface of the platform.

4. The stand of claim 1, wherein the treatment surface includes a plurality of ultraviolet light sources arranged laterally and/or horizontally along the treatment surface.

5. The stand of claim 1, wherein the at least one ultraviolet light source is configured to emit ultraviolet light having a peak wavelength in the ultraviolet-C range of ultraviolet radiation.

6. The stand of claim 1, wherein the at least one ultraviolet light source includes at least one light directing structure.

7. The stand of claim 1, further comprising a controller configured to operate the at least one ultraviolet light source to treat the wearable electronic device.

8. The stand of claim 1, wherein the platform further includes:
a second treatment surface located opposite the treatment surface, the second treatment surface having a second ultraviolet transparent structure; and
at least one second ultraviolet light source configured to direct ultraviolet radiation through the second transparent structure and onto a second surface of the wearable electronic device.

9. The stand of claim 8, wherein the second surface of the wearable electronic device is at least one of an inner surface of a band or a contact surface of the wearable electronic device.

10. The stand of claim 1, further comprising:
a base; and
a support member secured to the base, wherein the support member is secured to and supports the platform above a surface on which the base is located.

11. The stand of claim 1, further comprising at least one sensing device configured to acquire location data corresponding to a presence or an absence of the contact surface of the wearable electronic device on the outer surface of the transparent structure, wherein the stand is configured so that the at least one ultraviolet light source is off when the location data corresponds to an absence of the contact surface.

12. The stand of claim 1, further comprising at least one sensing device configured to acquire contamination data corresponding to a presence and/or amount of a contaminant on the contact surface of the wearable electronic device located on the outer surface of the transparent structure, wherein the stand is configured to adjust operation of the at least one ultraviolet light source based on the contamination data.

13. The stand of claim 1, further comprising at least one charging interface configured to enable a battery of the wearable electronic device to be charged while the wearable electronic device is located on the platform.

14. A stand for a wearable electronic device, the stand comprising:
a base configured for placement on a surface;
a support member secured to the base;
a platform configured to support the wearable electronic device, wherein the support member is secured to and supports the platform above the surface, wherein the platform includes a treatment surface having an ultraviolet transparent structure with an outer surface configured to conform to a shape of a contact surface of the wearable electronic device;
at least one ultraviolet light source configured to direct ultraviolet radiation through the outer surface of the transparent structure and onto the contact surface of the wearable electronic device; and
at least one charging interface configured to enable a battery of the wearable electronic device to be charged while the wearable electronic device is located on the platform.

15. The stand of claim 14, wherein the treatment surface includes a plurality of ultraviolet light sources arranged laterally and/or horizontally along the treatment surface.

16. The stand of claim 14, further comprising a controller configured to operate the at least one ultraviolet light source to treat the wearable electronic device.

17. The stand of claim 14, further comprising at least one sensing device configured to acquire location data corresponding to a presence or an absence of the contact surface of the wearable electronic device on the outer surface of the transparent structure, wherein the stand is configured so that the at least one ultraviolet light source is off when the location data corresponds to an absence of the contact surface.

18. A stand for a wearable electronic device, the stand comprising:
a platform configured to support the wearable electronic device, wherein the platform includes a treatment surface having an ultraviolet transparent structure with an outer surface configured to conform to a shape of a contact surface of the wearable electronic device;
at least one ultraviolet light source configured to direct ultraviolet radiation through the outer surface of the transparent structure and onto the contact surface of the wearable electronic device;
a controller configured to operate the at least one ultraviolet light source to treat the wearable electronic device; and
at least one sensing device configured to acquire location data corresponding to a presence or an absence of the contact surface of the wearable electronic device on the outer surface of the transparent structure, wherein the controller is configured to operate the at least one ultraviolet light source so that the at least one ultraviolet light source is off when the location data corresponds to an absence of the contact surface.

19. The stand of claim 18, further comprising at least one sensing device configured to acquire contamination data corresponding to a presence and/or amount of a contaminant on the contact surface of the wearable electronic device located on the outer surface of the transparent structure, wherein the controller is configured to adjust operation of the at least one ultraviolet light source based on the contamination data.

20. The stand of claim 18, further comprising at least one charging interface configured to enable a battery of the wearable electronic device to be charged while the wearable electronic device is located on the platform.
